(19) United States
(12) Patent Application Publication
(10) Pub. No.: US 2017/0273596 A1
(43) Pub. Date: Sep. 28, 2017

(54) METHOD FOR MONITORING SWALLOWING

(71) Applicant: NESTEC S.A., Vevey (CH)

(72) Inventors: Benjamin Le Reverend, Neuvecelle (FR); Philippe Pollien, Forel (CH); Florian Viton, Lausanne 26 (CH); Beatrice Aubert, Villars-Tiercelin (CH)

(21) Appl. No.: 15/508,694

(22) PCT Filed: Sep. 4, 2015
(86) PCT No.: PCT/EP2015/070233

§ 371 Date: Mar. 3, 2017

(30) Foreign Application Priority Data
Sep. 4, 2014 (EP) 14183564.5

(51) Int. Cl. A61B 5/08; A23L 33/00; A23L 29/269; A61B 8/08; A61B 5/00

(52) U.S. Cl. CPC A61B 5/082; A61B 8/08; A61B 8/488; A61B 5/4205; A23L 29/269; A23L 33/30; A23L 33/40; A23V 2002/00

(57) ABSTRACT

The present invention provides a method for monitoring swallowing in a subject, comprising: (a) providing a food product comprising a volatile compound to the subject; and (b) detecting release of the volatile compound in exhaled breath during and/or after swallowing of the food product.

### FIELD OF THE INVENTION

The present invention relates to the field of methods for monitoring a swallowing in a subject, for instance in the diagnosis of swallowing disorders or dysphagia.

### BACKGROUND OF THE INVENTION

Swallowing is a basic physiological function which is necessary for survival. Disorders in which subjects have difficulty swallowing are often associated with high mortality rates, due in part to starvation or dehydration. Failure to swallow properly may also lead to aspiration of food particles into the lungs, which often leads to pneumonia. Swallowing disorders may be referred to as dysphagia.

Conditions leading to dysphagia include, for example, oral cancer, stroke (cerebral infarction and haemorrhage) and craniocerebral trauma. A high proportion of such subjects develop dysphagia and subsequently aspiration pneumonia.

A number of methods are currently in clinical use in order to monitor swallowing in a subject. In X-ray video fluoroscopy, the subject swallows a food product containing a contrast medium. The swallowing process is then recorded as a video using fluoroscopy. For analysis, the video may be studied in slow motion.

Typically such a method may involve obtaining X-ray images over a period of 4-5 seconds at a rate of 15 images per second. This involves exposure to a significant dose of X-rays, with associated risks. The procedure is also technically complex and is not quantitative.

An alternative method is endoscopy, sometimes referred to as fiberoptic endoscopic examination of swallowing (FEES). By introducing a flexible endoscope through the nose of the subject, the ingestion of various food types can be studied. In some cases a fluorescent dye may be included in a fluid which is then swallowed, in order to facilitate visualization of residues in the oropharyngeal cavity. A disadvantage of endoscopy is that images cannot be obtained throughout the entire ingestion process, because the tongue and posterior pharyngeal wall tend to obscure the view during swallowing itself.

Another method for monitoring swallowing in real time is ultrasound sonography. However, this method is of limited use, in particular due to difficulties in detecting particular tissue structures and food residues.

Accordingly, there is a need for new methods for monitoring swallowing, for instance in order to diagnose dysphagia in a subject.

### SUMMARY OF THE INVENTION

The aim of the present invention is achieved by subject-matter specified in the independent claims. Particular embodiments of the invention are specified in the dependent claims.

Accordingly, in a first aspect the present invention provides a method for monitoring swallowing in a subject, comprising (a) providing a food product comprising a volatile compound to the subject; and (b) detecting release of the volatile compound in exhaled breath during and/or after swallowing of the food product.

In one embodiment the subject has, or is at risk of, dysphagia.

The volatile compound may be detected using mass spectrometry, a breath analyser/breathalyser or a microfluidics chip.

The volatile compound may be detected by a method selected from, for example, a group consisting of proton transfer reaction mass spectrometry either with a quadupole detector (PTR-MS) or time of flight PTR-TOF-MS), atmospheric-pressure chemical ionization mass spectrometry (APCI-MS), gas chromatography mass spectrometry (GC-MS) and gas chromatography ion-mobility mass spectrometry (GC-IMS).

Preferably, the volatile compound is detected by PTR-MS or PTR-TOF-MS.

The levels and the rate of depletion breath by breath of the volatile compound in exhaled breath after swallowing may be indicative of residues of the food product in the oropharyngeal cavity of the subject and/or indicative of aspiration of the food product by the subject.

The volatile compound may be selected from, for example, a group consisting of ethanol, limonene and ethyl butyrate.

Preferably, the volatile compound is ethanol.

The method may further comprise monitoring phases of the swallowing process in the subject. Preferably, the phases of the swallowing process are monitored by ultrasound imaging and/or ultrasound Doppler velocimetry.

In another aspect, the present invention provides a food product suitable for consumption by a dysphagic subject which contains an amount of a volatile compound detectable to enable monitoring of swallowing using the method defined herein.

The food product may have previously been spiked or sprayed with a volatile compound.

The food product may be, for example, a liquid, semi-solid or solid food product.

In one embodiment the food product is a thickened composition comprising a xanthan gum.

In one embodiment the food product comprises a food grade polymer capable of increasing an extensional viscosity of the nutritional composition.

The volatile compound may be selected from, for example, a group consisting of ethanol, limonene and ethyl butyrate.

Preferably, the volatile compound is ethanol.

In a further aspect, the present invention provides a use of a food product for monitoring swallowing in a subject, wherein the food product is a food product of present invention.

In a further aspect the present invention provides a food product according to the present invention for use in monitoring swallowing and/or diagnosing dysphagia in a subject.

Dysphagia may be diagnosed by a method according to the present invention.

In another aspect the present invention provides a use of a device suitable for detecting a volatile compound for monitoring swallowing in a subject. The device may be used in accordance with the method of the present invention.

In one embodiment the device is selected from a group consisting of a mass spectrometer, a breathalyser and a microfluidics chip.

### BRIEF DESCRIPTION OF THE FIGURES

FIGURE 1—Typical results obtained from the coupling of aroma release measured using PTR-TOF-MS with the different phases of oral processing. The circle marks a 5 mV trigger recorded using the analogue input.

### DETAILED DESCRIPTION OF THE INVENTION

Various preferred features and embodiments of the present invention will now be described by way of non-limiting examples.

The present invention relates in one aspect to a method for monitoring swallowing in a subject. The method advantageously permits the detection of both food residues in the oropharyngeal cavity as well as aspiration into the lungs. Moreover, the method may be quantitative, allows analysis both during and after swallowing itself and does not involve the risks associated with methods involving X-rays.

Monitoring Swallowing

The present method involves monitoring swallowing in a subject. By “monitoring swallowing” it is intended to include any method which involves studying the swallowing process, including the detection and diagnosis of disorders thereof (such as dysphagia). In particular, the method may be used to detect incomplete or partial swallowing (e.g. by detecting the presence of food residues in the oropharyngeal cavity) and/or aspiration.

The normal swallowing of a human (or mammal) involves three distinct phases which are interdependent and well-coordinated: (i) the oral, (ii) the pharyngeal, and (iii) the oesophageal phases. In the oral phase, which is under voluntary control, food that has been chewed and mixed with saliva is formed into a bolus for delivery by voluntary tongue movements to the back of the mouth, into the pharynx. The pharyngeal phase is involuntary and is triggered by food/liquid bolus passing through the faucial pillars into the pharynx. Contraction of the three constrictors of the pharynx propels the bolus towards the upper oesophageal sphincter. Simultaneously, the soft palate closes the nasopharynx. The larynx moves upwards to prevent food or liquid passing into the airway, which is aided by the backward tilt of the epiglottis and closure of the vocal folds. The oesophageal phase is also involuntary and starts with the relaxation of the upper oesophageal sphincter followed by peristalsis, which pushes the bolus down to the stomach.

The method of the present invention may involve monitoring the phases of the swallowing process in a subject. As used herein ‘monitoring of the phases of the swallowing process’ is synonymous with observing or visualising the phases of the swallowing process.

Monitoring of the phases of the swallowing process in a subject may be performed using any device or method which enables the phases of the swallowing process to be observed. For example, the swallowing process may be observed using magnetic resonance imaging (MRI), ultrasound imaging and/or ultrasound Doppler velocimetry techniques.

Dysphagia

The method of the present invention may be used to monitor swallowing in a subject having, or at risk of having, a medical condition which causes difficulty in swallowing.

Dysphagia refers to the symptom of difficulty in swallowing. General causes of dysphagia have been identified and include, but are not limited to, a decreased ability to swallow, the tongue not exerting enough pressure on the soft palate, abnormal epiglottis behavior, etc. The consequences of untreated or poorly managed oral pharyngeal dysphagia can be severe, including dehydration, malnutrition leading to dysfunctional immune response, and reduced functionality, airway obstruction with solid foods (choking), and airway aspiration of liquids and semi-solid foods, promoting aspiration pneumonia and/or pneumonitis.

Oesophageal dysphagia affects a large number of individuals of all ages, but is generally treatable with medications and is considered a less serious form of dysphagia. Oesophageal dysphagia is often a consequence of mucosal, mediastinal, or neuromuscular diseases. Mucosal (intrinsic) diseases narrow the lumen through inflammation, fibrosis, or neoplasia associated with various conditions (e.g. peptic stricture secondary to gastrooesophageal reflux disease, oesophageal rings and webs [e.g. sideropenic dysphagia or Plummer-Vinson syndrome], oesophageal tumors, chemical injury [e.g., caustic ingestion, pill esophagitis, sclerotherapy for varices], radiation injury, infectious esophagitis, and eosinophilic esophagitis). Mediastinal (extrinsic) diseases obstruct the oesophagus by direct invasion or through lymph node enlargement associated with various conditions (tumors [e.g., lung cancer, lymphoma], infections [e.g., tuberculosis, histoplasmosis], and cardiovascular [dilated auricula and vascular compression]). Neuromuscular diseases may affect the oesophageal smooth muscle and its innervation, disrupting peristalsis or lower oesophageal sphincter relaxation, or both, commonly associated with various conditions (achalasia [both idiopathic and associated with Chagas disease], scleroderma, other motility disorders, and a consequence of surgery [i.e., after fundoplication and ant reflux interventions]). It is also common for individuals with intraluminal foreign bodies to experience acute oesophageal dysphagia.

Oral pharyngeal dysphagia, on the other hand, is a very serious condition and is generally not treatable with medication. Oral pharyngeal dysphagia also affects individuals of all ages, but is more prevalent in older individuals. Worldwide, oral pharyngeal dysphagia affects approximately 22 million people over the age of 50. Oral pharyngeal dysphagia is often a consequence of an acute event, such as a stroke, brain injury, or surgery for oral or throat cancer. In addition, radiotherapy and chemotherapy may weaken the muscles and degrade the nerves associated with the physiology and nervous innervation of the swallow reflex. It is also common for individuals with progressive neuromuscular diseases, such as Parkinson's Disease, to experience increasing difficulty in swallowing initiation. Representative causes of oropharyngeal dysphagia include those associated neurological illnesses (brainstem tumors, head trauma, stroke, cerebral palsy, Guillain-Barre syndrome, Huntington's disease, multiple sclerosis, polio, post-polio syndrome, metabolic encephalopathies, amyotrophic lateral sclerosis, Parkinson's disease, dementia), infectious illnesses (diphtheria, botulism, Lyme disease, syphilis, mucositis [herpetic, cytomegalovirus, Candida, etc.]), autoimmune illnesses (lupus, scleroderma, Sjogren's syndrome), metabolic illnesses (amyloidosis, Cushing's syndrome, thyrotoxicosis, Wilson's disease), myopathic illnesses (connective tissue disease, dermatomyositis, myasthenia gravis, myotonic dystrophy, oculopharyngeal dystrophy, polymyositis, sarcoidosis, paraneoplastic syndromes, inflammatory myopathy), iatrogenic illnesses (medication side effects [e.g., chemotherapy, neuroleptics, etc.], post surgical muscular or neurogenic, radiation therapy, corrosive [pill injury, intentional]), Tardive Dyskinesia [A chronic disorder of the nervous system characterized by involuntary jerky movements of the face, tongue, jaws, trunk, and limbs, usually developing as a late side effect of prolonged treatment with antipsychotic drugs], and structural illnesses (cricopharyngeal bar, Zenker's diverticulum, cervical webs, oropharyngeal tumors, osteophytes and skeletal abnormalities, congenital [cleft palate, diverticulae, pouches, etc.]).

The method of the present invention may be used to monitor swallowing in a subject who has, or is a risk of, any one or more of the conditions recited above.

Dysphagia is not generally diagnosed although the disease has major consequences on patient health and healthcare costs. Individuals with more severe dysphagia generally experience a sensation of impaired passage of food from the mouth to the stomach, occurring immediately after swallowing. Among community dwelling individuals, perceived symptoms may bring patients to see a doctor. Among institutionalized individuals, health care practitioners may observe symptoms or hear comments from the patient or his/her family member suggestive of swallowing impairment and recommend the patient be evaluated by a specialist. As the general awareness of swallowing impairments is low among front-line practitioners, dysphagia often goes undiagnosed and untreated. Yet, through referral to a swallowing specialist (e.g., speech language pathologist), a patient can be clinically evaluated and dysphagia diagnosis can be determined.

Severity of dysphagia may vary from: (i) minimal (perceived) difficulty in safely swallowing foods and liquids, (ii) an inability to swallow without significant risk for aspiration or choking, and (iii) a complete inability to swallow. Many people with swallowing impairment do not seek medical care when symptoms are mild or unrecognized. For example, “silent aspiration,” a common condition among elderly, refers to the aspiration of the oropharyngeal contents during sleep. People may compensate for less-severe swallowing impairments by self-limiting the diet. The aging process itself, coupled with chronic diseases such as hypertension or osteoarthritis, predisposes elderly to (subclinical) dysphagia that may go undiagnosed and untreated until a clinical complication such as pneumonia, dehydration, malnutrition (and related complications) occurs. Yet, the differential diagnosis of ‘aspiration pneumonia’ is not necessarily indicated as a result of current care practices.

Aspiration

The term “aspiration” refers to the drawing of a foreign substance into the respiratory tract. Particularly, as used herein, aspiration refers to the drawing of a food product into the respiratory tract during swallowing.

Aspiration can occur before, during, or after the swallow. Aspiration occurs before the swallow in the case of a delayed or absent initiation of the swallow. It may also be the result of poor tongue control, which allows food to trickle into the pharynx while the patient is still chewing. Aspiration occurs during the swallow when the vocal folds fail to adduct or the larynx fails to elevate. Aspiration can occur after the swallow in several different circumstances: the patient may pocket food in the oral cavity, food may get stuck in the pharyngeal recesses or due to reduced laryngeal elevation, food may remain on top of the larynx.

Subject

The term “subject” as used herein is interchangeable with “patient” or “individual”. The term subject may refer to any animal, mammal or human having or at risk for a medical condition that can benefit from a method of monitoring swallowing as provided by the present invention.

For example the subject may have, or be at risk of, a condition associated with dysphagia.

Food Product

The present invention involves providing a food product comprising a volatile compound to the subject; and detecting release of the volatile compound in exhaled breath during and/or after swallowing of the food product.

The present invention also provides a food product suitable for consumption by a dysphagic subject which contains an amount of a volatile compound detectable to enable monitoring of swallowing.

A food product which comprises a volatile compound and is suitable for consumption by a dysphagic subject preferably has essentially the same swallowing properties as a food product that would be prescribed by a healthcare practitioner in order to avoid a clinical problem such as difficulty swallowing, residues in the oropharyngeal cavity or aspiration (e.g. ThickenUPClear™). Inclusion of a volatile compound preferably does not modify the textural properties of the food products which have been specifically tailored to aid safe swallowing. The food product may have previously been spiked or sprayed with a volatile compound. ‘Spiked’ or ‘sprayed’ is used herein to describe the addition of a volatile compound to the food product. The volatile compound may be essentially absent from the food product prior to the addition via spiking or spraying.

Spiking describes that the volatile compound is added within/into the food product. Spraying describes that the volatile compound coats all/or part of the surface of the food product.

The food product may be solid or liquid, but is preferably a solid or semi-solid food product.

In certain embodiments the food product may be a thickened liquid or a puree of solid foods, both of which have been shown to be the most effective means of preventing choking and aspiration during the eating process. Thickened liquids are designed to have three properties: (i) a more cohesive bolus that can be maintained throughout the action of swallowing, (ii) slower delivery to the throat, thereby compensating for the increased period in which the swallowing reflexes prepare for the thickened liquid, and (iii) provide greater density to increase awareness of the presence of food or liquid bolus in the mouth.

The food product may be water, milk, soup, yogurt, orange juice, coffee, tea, soda, or combinations thereof.

In some embodiments, the food product as described above may comprise starch or gum thickeners (thickening product). For example, the food product may be a beverage or liquid food which comprises a starch or gum thickener. The presence of starch or gum thickeners increases the viscosity of the beverage or liquid food and thus aids swallowing.

In certain embodiments, the volatile compound may be provided in the thickening product.

Examples of thickening products which may be used to thicken a food product of the present invention are described in WO 2013/160207, WO 2013/087916 and WO 2013/087918 (each of which is herein incorporated by reference).

Briefly, WO 2013/160207 describes a thickened composition having a xanthan gum thickening component, and orally administering the composition to an individual having, or at risk of having, a swallowing impairment. It is described that the administration of a thickened composition including a xanthan gum thickening component increases the efficacy of a swallow response by decreasing the presence of pharyngeal residue while at least maintaining swallowing safety. The xanthan gum is food grade and can be commercially obtained from numerous suppliers. Xanthan gum is a high molecular weight, long chain polysaccharide composed of the sugars glucose, mannose, and glucuronic acid.

The backbone is similar to cellulose, with added side chains of trisaccharides. The compositions contain xanthan gum in an amount ranging from about 0.5 g to about 8 g, about 1 g to about 7 g, about 2 g to about 6 g, or about 3 g to about 4 g, per every 100 mL of a liquid carrier (e.g., water). In an embodiment, the compositions contain xanthan gum in an amount ranging from about 1.2 g to about 6 g. Thickening compositions comprising xanthan gum are available commercially, for example NestléHealthScience Resource® ThickenUPClear™.

Thus, in some embodiments the food product comprises a thickening composition having a xanthan gum thickening component. The food product may consist of a thickening composition having a xanthan gum thickening component. The xanthan gum thickening component may comprise the volatile compound.

The food product may comprise or consist of a ThickenUPClear™ food product.

WO 2013/087916 describes nutritional products having improved cohesiveness of food boluses. The nutritional products may include nutritional compositions and high molecular weight, water-soluble polymers such that the nutritional products have extensional viscosities that provide improved cohesiveness to the nutritional products and Trouton ratios of at least 6. The method for making such a nutritional composition comprises providing a nutritional composition and adding a food grade polymer to the nutritional composition to form a nutritional product having a Trouton ratio that is at least 6. The food grade polymer may be selected from plant extracted gums, plant-derived mucilages and combinations thereof. The plant extracted gums may furthermore be selected from okra gum, konjac mannan, tara gum, locust bean gum, guar gum, fenugreek gum, tamarind gum, cassia gum, acacia gum, gum ghatti, pectins, cellulosics, tragacanth gum, karaya gum, or any combinations thereof. Further, the plant-derived mucilages may be selected from the group consisting of kiwi fruit mucilage, cactus mucilage (Ficus indica), psyllium mucilage (Plantago ovata), mallow mucilage (Malva sylvestris), flax seed mucilage (Linum usitatissimum), marshmallow mucilage (Althaea officinalis), ribwort mucilage (Plantago lanceolata), mullein mucilage (Verbascum), cetraria mucilage (Lichen islandicus), or any combinations thereof.

Thus in certain embodiments of the present invention, the food product may comprise water-soluble polymers such that the food product has improved cohesiveness and a Trouton ratio of at least 6.

Volatile Compound

The food product of the present invention comprises a volatile compound.

The volatile compound may be any volatile compound which can be detected in the method according to the first aspect of the invention. The volatile compound may be any compound which can be detected in the exhaled breath of a subject during and/or after swallowing.

The “volatile compound” may be a compound with a high vapour pressure at room temperature. The volatile compound can be released from a matrix and can be found in the headspace surrounding the product. This high vapour pressure results from a low boiling point and/or a low solubility in the matrix, which causes large numbers of molecules to evaporate or sublimate from the liquid or solid form of the compound and enter the surrounding air. Molecules which enter the surrounding air may be referred to as the gas fraction.

Preferably, the volatile compound is non-toxic at the levels at which it is administered to the subject.

Preferably, the volatile compound is present in the food product at a concentration which yields gas fractions in the part per trillion (ppt), parts per billion (ppb), parts per million (ppm) range or parts per thousand (‰).

The food product may comprise at least one, at least two, at least three, at least four or at least five or more volatile compound as defined herein. The food product may comprise one, two, three, four, five or more volatile compounds.

In certain embodiments, the volatile compound is selected from a group consisting of ethanol, limonene and ethyl butyrate.

The volatile compounds may be incorporated into a food product as flavour compounds. The volatile compound may occur naturally in fruits (e.g. orange). Volatile compounds cover a range of volatility and lipophilicity. Table 1 below shows the volatility and lipophilicity of exemplified volatile compounds (database Episuite 4.1).

| | | Lipophilicity: | Volatility: |
|---|---|---|---|
| | | Oil/Water parti- | Air/water parti- |
| | | tion coefficient | tion coefficient |
| Compounds | CAS | log Kow [w/v] | log K [w/v] |
| Ethanol | 64-17-5 | −0.31 | −3.69 |
| Vanillin | 121-33-5 | 1.05 | −8.47 |
| 2-Methylbutanal | 96-17-3 | 1.23 | −2.20 |
| 3-Methylbutanal | 590-86-3 | 1.23 | −2.19 |
| 2-Methylpropanal | 78-84-2 | 0.74 | −2.31 |
| 2,3-Pentanedione | 600-14-6 | −0.85 | −4.97 |
| Isoamylacetate | 123-92-2 | 2.26 | −1.65 |
| 2,3-Butanedione | 432-03-8 | −1.34 | −5.09 |
| Acetaldehyde | 75-07-0 | −0.17 | −2.56 |
| Ethylbutyrate | 105-54-4 | 1.85 | −1.79 |
| Methanethiol | 74-93-1 | 0.78 | −0.97 |
| Dimethylsulfide | 75-18-3 | 0.92 | −1.49 |
| Limonene | 138-86-3 | 4.38 | 0.12 |

In one embodiment, the volatile compound may have an air/water partition coefficient (log K [w/v]) of −10 to +1. For example the volatile compound may have an air/water partition coefficient (log K [w/v]) of −5 to +1.

In one embodiment, the volatile compound may have an oil/water partition coefficient (log Kow [w/v]) of −2 to +5.

Preferably, the volatile compound is ethanol.

In certain embodiments the concentration of ethanol in the food product is similar to that which occurs naturally in orange juice. For example, the concentration of ethanol may be, for example 0.05% to 0.5%. Preferably, the concentration of ethanol is 0.1%.

The food product may comprise a polar and a non-polar volatile compound.

The term ‘polar’ is used herein according to its conventional meaning to refer to a molecule having an electric dipole or multipole moment.

Polar compounds are hydrophilic and highly soluble in water e.g. ethanol. Non-polar compounds are lipophilic and have low solubility in water e.g. limonene.

The volatile compound may be spiked into or sprayed onto the food product.

In certain embodiments, the present invention provides the advantage that only low levels of the volatile compound in the food product are required, compared for example to the level of a fluorescent dye may be included in a fluid to facilitate FEES.

Preferably, the volatile compound is spiked into or sprayed onto the food product at a known level/amount/concentration such that the level of release of the volatile compound in exhaled breath following swallowing of the food product can be compared between different tests. For example the level of release may be compared between a test and a control subject or between tests performed on the same subject on different occasions (see below).

Detecting Release

The method of the present invention is suitable for detecting the release of the volatile compound in exhaled breath during and/or after swallowing of the food product.

The detection may be performed using any method or apparatus which is suitable for determining the level of the volatile compound present in the exhaled breath (e.g. any method or apparatus that can determine if the volatile compound is present in the exhaled breath and/or if the level of volatile compound is decreasing in subsequent exhalations and/or the rate at which the level of volatile compound detected in exhaled breath decreases).

Methods and apparatus which are suitable for detecting the release of the volatile compound are well-known in the art. For example, in some embodiments the volatile compound may be detected using mass spectrometry, a breathalyser or a microfluidics chip.

In certain embodiments the release of the volatile compound is detected by mass spectrometry. For example, the release may be detected by a method selected from a group consisting of proton transfer reaction mass spectrometry (PTR-MS or PTR-TOF-MS), atmospheric-pressure chemical ionization mass spectrometry (APCI-MS), and gas chromatography ion-mobility mass spectrometry (GC-IMS).

The volatile compound may be detected on-line in real time by a time resolved method selected from, for example, a group consisting of PTR-MS, PTR-TOF-MS, APCI-MS, and GC-IMS.

In certain embodiments, the release of the volatile compound is detected by PTR-MS or PTR-TOF-MS.

In certain embodiments, the release of the volatile compound is detected by GC-IMS.

The volatile compound may be detected by ‘nosespace’ analysis, which refers to the detection of volatile compound in breath exhaled from the nose. The volatile compound may be detected by ‘mouthspace’ analysis, which refers to the detection of volatile compound in breath exhaled from the mouth.

Breathalysers/breath analysers which detect volatile compounds in the exhaled breath of a subject are well known in the art, for example to detect ethanol. Such breathalysers are described, for example, in EP1584924, U.S. Pat. No. 4,770,026 and WO 2010/009406 (each of which is hereby incorporated by reference). The skilled person will appreciate that such devices are suitable for use in a method according to the first aspect of the invention. In addition, such breathalysers can be readily modified in order to detect alternative volatile compounds which may be detected in the method according to the present invention.

Microfluidics devices for assaying components of exhaled breath are also known in the art (see Li et al; Anal Chem. 2012 Feb. 7; 84(3):1288-93; Fu et al; Cancer Med. 2014 February; 3(1):174-81 and https://www.lcaos.eu)

The detection of the volatile compound in exhaled breath after swallowing may be indicative of residues of the food product in the oropharyngeal cavity and/or indicative of aspiration of the food product by the subject. Such detection is indicative that the subject has problems swallowing.

In certain embodiments the level of volatile compound detected in the exhaled breath of a subject during and/or after swallowing may be compared to a control level.

Reference to a “control” broadly includes data that the skilled person would use to facilitate the accurate interpretation of technical data. As such “control level” is interchangeable with “reference level”. In an illustrative example, the level or levels of volatile compound in the exhaled breath of a subject are compared to the respective level or levels of the same volatile compound in one or more cohorts (populations/groups) of control subjects selected from a subject cohort wherein the subjects have been diagnosed with a condition which causes difficulty in swallow (e.g. dysphagia) and a subject cohort wherein the subjects have been predetermined not to have any condition which causes difficulty swallowing (e.g. dysphagia).

The control level may represent the level of volatile compound detected in the exhaled breath of a control cohort, wherein the same food product and same volatile compound are administered to the test subject and the control subjects. As the skilled person will recognise, the total amount and concentration of the food product and volatile compound (along with all other variables) should be kept as consistent as possible between the test subject and control subject(s).

In some embodiments, the control may be the level of volatile compound in a sample from the test subject taken at an earlier time point. Thus, a temporal change in the level of the volatile compound can be used to identify difficulty swallowing or provide a correlation as to the subject's ability to swallow.

In some embodiments, control or reference levels for the detection of a given concentration of a particular volatile compound, administered in a given food product, may be stored in a database and used in order to interpret the results of the method as performed on the subject.

Inefficient swallowing or dysphagia may be associated with;
i) increased levels of volatile compound detected in exhaled breath during and/or after swallow;
ii) levels of volatile compound which are detectable in an increased number of exhalations following swallow;
iii) levels of volatile compound which are detectable for a longer time period following swallow;
iv) a more gradual decrease in levels of volatile compound detectable in subsequent exhalations following swallow (e.g rate of depletion breath by breath); and/or
v) an increased time period between the initiation of swallowing and the detection of volatile compound in the first exhalation following swallow;

in comparison to reference/control levels in a subject/cohort with efficient swallowing.

The level of volatile compound may be quantified by amplitude or area under the curve methodologies or by calculating the level and/or ratio of the volatile compound compared to a reference compound.

Residues

The term “residues” refers to deposits of the food product which remain present in the subject's oropharyngeal cavity after swallowing or are aspirated into the subject's respiratory tract. Volatile compound which is present on/in the food product will be released from the deposits of food product which remain present in the oropharyngeal cavity, or are aspirated, and can be detected by the method of the present invention.

As a simple illustration, the more residues there are in the oropharyngeal cavity, the more volatile compound will be released, as a function of the residues surface area and volatile partitioning.

In certain embodiments, volatile compound released from aspirated residues are detected by the method of the present invention.

Use

The present invention further provides a use of a food product for monitoring swallowing in a subject. In certain embodiments, the subject has, or is at risk of dysphagia.

Preferably, the food product is a food product as defined herein.

The present invention also provides a use of a device suitable for detecting a volatile compound for monitoring swallowing in a subject.

The term “device” refers to any analytical instrument or machine which is suitable for detecting the presence of the volatile compound in the exhaled breath of the subject.

The device may be any device which is suitable for detecting the release of a volatile compound in exhaled breath of a subject during and/or after swallowing.

The device may be any device as described herein.

### EXAMPLES

### Example 1

### Detection of Volatile Compounds in Breath Products Used

A commercial orange juice (Eckes-Granini Group GmbH, Nieder-Olm, Germany) was chosen due to the high content of volatile terpenes in orange juice and strong volatile signal obtained by nosespace analysis. Two major compounds were followed and tentatively identified at m/z 47.0494, (C₂H₆O)H⁺ corresponding mainly to ethanol and m/z 137.1325 corresponding mainly to limonene. These identifications were confirmed by an off-line measurement using static headspace GC-MS.

In Vivo Aroma Release

Assessor exhaled air was sampled via two glass tubes inserted into the nostril and fixed on laboratory glasses [1]. This tailor-made nosepiece allowed the subject to breathe comfortably during eating or drinking. The majority of the breath-air was released into the room. Only 80 ml/min was drawn into the PTR-TOF-MS (Ionicon, Austria) via its transfer line connected to the nosepiece. To avoid condensation, the transfer line was heated at 100° C. A ⅛ inch copper tubing of 20 cm length was inserted inside the PTR-TOF-MS transfer line and around its 1/16 inch inlet capillary peek tubing passing the heated transfer line. Due to the high copper thermal conductivity it was possible to heat the capillary peek tubing until its extremity.

The PTR-TOF-MS was set-up to monitor a full spectrum from m/z 10 to 350 every 0.1 s. Internal mass scale calibration was done on a parasitic ion always present, m/z 29.9974(NO)⁺ and acetone coming from usual air lab contamination and, as body metabolite, also present in breath air at m/z 59.0491 (C₃H₆O)H⁺.

Assessor breathing pattern were also followed on m/z 59.0491 (C₃H₆O)H⁺ corresponding to acetone.

In Vivo Oral Processing

UltraSound imaging was acquired using a Siemens SC2000 (Siemens, Renens, Switzerland) used in parallel to observe in real time the drinking process by maintaining the ultrasonic probe under the oral cavity.

To synchronize precisely the acquisition time of the PTR-TOF-MS and the UltraSound instrument, a 5 mV trigger (amplified to 1.6V for the PTR-TOF-MS) was recorded on analog input of both instruments (analogue input in PTR-TOF-MS and ElectroCardioGraphy input for the Ultrasound).

Results

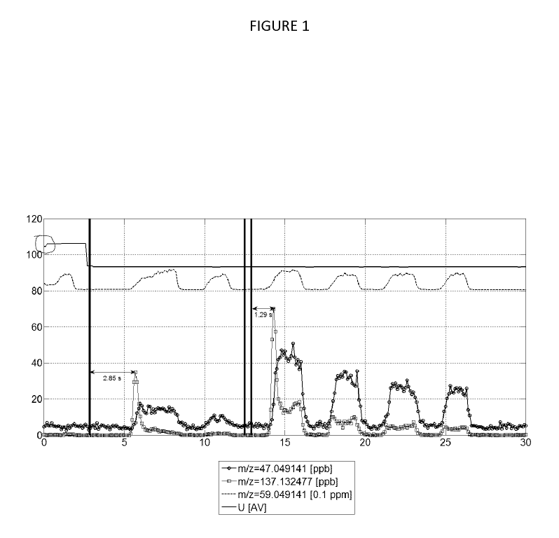

Using this setup, the inventors were able to determine the presence of volatile compounds in the subject's breath. In FIGURE 1, one can see that three events (first contact in mouth, swallowing start, swallowing end, all materialized by thick vertical lines) were identified using the ultrasound images. First the product inlet in the mouth was recorded around 2.5 s after the beginning of the experiment as normalized using the two equipments. The first peak of limonene was then measured 2.85 s after the first contact of the product in mouth. Limonene (m/z 137.1325) quickly rarefies in the nose space prior to swallowing, whilst ethanol (m/z 47.0491) sustains its intensity over two breath cycles prior swallowing. The initiation of swallowing and completion of swallowing are very close to one another (Δt=0.2 s) and result in no aroma released during the swallowing phase itself 1.29 s after the completion of the swallowing phase, a new burst of limonene can be measured, which again quickly rarefies whilst ethanol rarefaction in the nose space is much slower. This period of 1.29 s of delay between the two signals gives us a first approximation of the time spent by the volatiles in the pharynx before exhalation.

### Example 2

### Detection of Volatile Compounds in Breath for Thickened Liquids Products Used

A commercial orange flavoured syrup was used to flavour the compositions due to the high content of volatile terpenes in orange fruits and strong volatile signal obtained by nosespace analysis. A mass of 20 g was used in all compositions for this example so that a comparison could be made between products. Two major compounds were followed and tentatively identified at m/z 47, (C₂H₆O)H³⁰ corresponding mainly to ethanol and m/z 81 corresponding to a limonene fragment. The m/z values differ between Example 1 and Example 2 due to technological differences existing between the PTR-MS and the PTR-TOF-MS.

To increase the level of viscosity of the products tested, we used two thickeners; either 65 g or 75 g of sugar molasses were used in the final liquid composition which gives a Newtonian viscosity profile or either 0.6 g or 1.8 g of Resource ThickenUP Clear were used in the final liquid composition which gives a non-Newtonian, shear thinning, viscosity profile to the product such as those usually used for the treatment of dysphagia. For all compositions Vittel water was added to the mixture so that the total volume was 100 m1.

In Vivo Aroma Release

Assessor exhaled air was sampled via two glass tubes inserted into the nostril and fixed on laboratory glasses [1]. This tailor-made nosepiece allowed the subject to breathe comfortably during eating or drinking. The majority of the breath-air was released into the room. Only 80 ml/min was drawn into the PTR-MS (Ionicon, Austria) via its transfer line connected to the nosepiece. To avoid condensation, the transfer line was heated at 100° C. A ⅛ inch copper tubing of 20 cm length was inserted inside the PTR-MS transfer line and around its 1/16 inch inlet capillary deactivated stainless steel tubing passing the heated transfer line. Due to the high copper thermal conductivity it was possible to heat the capillary tubing until its extremity.

The PTR-MS was set-up to monitor selected masses m/z 37 (water vapour), m/z 47 (ethanol) and m/z 81 (limonene) every 0.12 s.

Assessor breathing pattern were also followed on m/z 37 corresponding to the water vapour present in the exhaled breath. This signal for m/z 37 was used in order to defined the beginning and end of each exhalation cycles, using an automated routine analysis developed using the MATLAB 2013b (Then Mathworks inc.) software. After swallowing the maximum value in the limonene signal intensity (I) was extracted for each one of the first five peaks, and the median time (t) was extracted for each one of the first five peaks. In order to assess the effect of viscosity on the persistence of the aroma in the exhaled breath the slope of the curve log(I)=a.log(t) was fitted using the polyfit function og MATLAB 2013b.

Results

Using this setup, the inventors were able to determine the presence of volatile compounds in 11 healthy subjects' breath. The characteristic slope (a) of the signal log(I)=a.log(t) was always negative since the signal decays after swallowing as shown on FIGURE 1.

Increasing the viscosity had the effect to decrease the maximum intensity of the signal, but most importantly the signal did not decay as fast as for a non-thickened control sample, prolonging aroma release such that the mean calculated slope (a) across subjects was less negative.

TABLE 1
Formulations of the compositions used in Example 2 and corresponding
slopes of decay measured in exhaled breath.

| Ingredient\Product | Control | M65 | M75 | TUC06 | | TUC18 | |
|---|---|---|---|---|---|---|---|
| Molasses | 0 | 65 g | 75 g | 0 | | 0 | |
| ThickenUPClear ® | 0 | 0 | 0 | 0.6 | g | 1.8 | g |
| Orange syrup | 20 g | 20 g | 20 g | 20 | g | 20 | g |

| | | | | | |
|---|---|---|---|---|---|
| Mean Calculated | −3.31 | −2.32 | −2.34 | −2.77 | −2.62 |
| Slope | | | | | |

Using a 2-way ANOVA test, it was found that there were significant differences between the control and the M65 and M75 products (p<0.06).

[1] Santo Ali, Philippe Pollien, Christian Lindinger and Chahan Yeretzian, in vivo analysis of aroma release while eating food: a novel set-up for monitoring on-line nosespace air, 1st International Conference on Proton Transfer Reaction Mass Spectrometry and Its Applications, 161-164, (2003).

All publications mentioned in the above specification are herein incorporated by reference. Various modifications and variations of uses and methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in the relevant fields, are intended to be within the scope of the following claims.

1. A method for monitoring swallowing in a subject, comprising:
providing a food product comprising a volatile compound to the subject; and
detecting release of the volatile compound in exhaled breath during and/or after swallowing of the food product.

2. A method according to claim 1 wherein the subject has, or is at risk of, dysphagia.

3. A method according to claim 1, wherein the volatile compound is detected using a method selected from the group consisting of mass spectrometry, a breath analyser/breathalyser and a microfluidics chip.

4. A method according to claim 1, wherein the volatile compound is detected by a method selected from a group consisting of proton transfer reaction mass spectrometry (PTR-MS or PTR-TOF-MS), atmospheric-pressure chemical ionization mass spectrometry (APCI-MS), gas chromatography mass spectrometry (GC-MS) and gas chromatography ion-mobility mass spectrometry (GC-IMS).

5. A method according to claim 1, wherein levels of the volatile compound in exhaled breath after swallowing are indicative of residues of the food product in the oropharyngeal cavity of the subject.

6. A method according to claim 1, wherein levels of the volatile compound in exhaled breath after swallowing are indicative of aspiration of the food product by the subject.

7. A method according to claim 1, wherein the volatile compound is selected from the group consisting of ethanol, limonene and ethyl butyrate.

8. A method according to claim 7, wherein the volatile compound is ethanol.

9. A method according to claim 1 which further comprises monitoring phases of the swallowing process in the subject.

10. A method according to claim 9 wherein the phases of the swallowing process are monitored by ultrasound imaging and/or ultrasound Doppler velocimetry.

11. A food product suitable for consumption by a dysphagic subject which contains an amount of a volatile compound detectable to enable monitoring of swallowing in the subject, the volatile compound being exhaled in breath during and/or after swallowing of the food product.

12. A food product according to claim 11 which has been spiked or sprayed with a volatile compound.

13. A food product according to claim 11 wherein the food product is a liquid, semi-solid or solid food product.

14. A food product according to claim 11 wherein the food product is a thickened composition comprising a xanthan gum.

15. A food product according to claim 11 wherein the food product comprises a food grade polymer capable of increasing an extensional viscosity of the nutritional composition.

16. A food product according to claim 11 wherein the volatile compound is selected from the group consisting of ethanol, limonene and ethyl butyrate.

17. A food product according to claim 16 wherein the volatile compound is ethanol.

18-19. (canceled)

20. A food product according to claim 11 for use in monitoring swallowing and/or diagnosing dysphagia in a subject.

21. (canceled)

22. A method for monitoring swallowing in a subject comprising using a device which can detect a volatile compound exhaled by a subject during ingestion of the volatile compound.

23. A use according to claim 22, wherein the device is selected from the group consisting of a mass spectrometer, a breathalyser and a microfluidics chip.
